(19) United States
(12) Patent Application Publication
(10) Pub. No.: US 2011/0150837 A1
(43) Pub. Date: Jun. 23, 2011

(54) Amphiphilic polymer functionalized by methionine

(75) Inventors: You-Ping CHAN, Ternay (FR); Corine Vialas, Irigny (FR)

(73) Assignee: Flamel Technologies, Venissieux (FR)

(21) Appl. No.: 12/977,816

(22) Filed: Dec. 23, 2010

Related U.S. Application Data
(60) Provisional application No. 61/289,709, filed on Dec. 23, 2009.

(30) Foreign Application Priority Data
Dec. 23, 2009 (FR) 09 59460
Dec. 23, 2010 (IB) PCT/IB2010/056041

(51) Int. Cl. A61K 47/42; C07K 2/00; C08F 8/34; A61K 38/27; A61K 38/21; A61K 47/32; B82Y 5/00

(52) U.S. Cl. 424/85.7; 530/345; 525/329.8; 514/11.3; 977/773

(57) ABSTRACT

The present invention relates to new amphiphilic polymers comprising hydrophobic groups and methionine groups.

It also relates to compositions with a controlled release profile comprising such polymers, non-covalently combined with an active ingredient, in particular an active ingredient such as a peptide or a protein comprising in their sequence at least one amino acid which is sensitive to oxidation.

The present invention relates to new amphiphilic polymers with a hydrophilic backbone and bearing hydrophobic groups including methionine groups.

Generally, the amphiphilic polymers are capable, depending on their chemical structures, of forming nanoparticles of variable size in aqueous medium. Thus, polymers the backbone of which is linear, hydrophilic and bears hydrophobic side groups, can easily form nanoparticles in water. These nanoparticles, depending on the base material used for their production, may be biocompatible and optionally biodegradable. They are used in particular in the field of medicine for the vectorization of medicaments.

In this field, the applicant company has for some ten years been developing nanoparticulate and microparticulate systems based on polyamino acids comprising various hydrophobic groups and known as Medusa®.

Thus, FLAMEL TECHNOLOGIES' patent application WO 2003/104303 describes glutamic acid polymers comprising alpha-tocopherol grafts which, in water, form nanoparticles capable of combining insulin. The publication Y P. Chan et al. Expert Opin. Drug. Deliv. 2007, 4(4) 441-451, describes that formulations produced with these nanoparticles and proteins such as interferon-alpha or interleukin 2 make it possible, after a sub-cutaneous injection in humans, to obtain measurable plasmatic concentrations for a period at least equal to a week. Similarly, it is known that amphiphilic polymers, based on pullulan and comprising cholesterol grafts assemble together in aqueous medium in order to form nanoparticles which are able to reversibly combine proteins such as insulin (Akiyoshi et al. J. Controlled Release 1998, 54, 313-320). Other analogous polymers based on chitosan or dextran have been developed in particular in order to obtain hydrogel films or implants (WO 00/14155).

As is clear from the above, the uses of amphiphilic polymers in the field of protein and peptide formulation are numerous.

Unfortunately, obtaining amphiphilic polymer/protein formulations which are stable in aqueous medium for at least two years at 5° C. remains a major challenge, in particular as regards the vulnerability of certain therapeutic proteins as regards oxidation.

In fact, it is now widely recognized that certain amino acids such as methionine, cysteine, histidine or tryptophan, components of numerous proteins or enzymes, can oxidize during the formulation of the latter, or even over time. For obvious reasons, this oxidation phenomenon can have harmful consequences on the biological activity of the active ingredients containing these amino acids (Cleland et al. Crit. Rev. Ther. Drug Carrier Systems 1993, 10, 307-377).

In order to prevent this undesirable oxidation, in 1993 Takruri (U.S. Pat. No. 5,272,135) proposed utilizing proteins or enzymes comprising methionine in their sequence, by formulating them with methionine. This concept is now well-known to a person skilled in the art working in the field of the stabilization of formulations containing proteins. For example, the patent application WO 2008/145323 describes an interferon alpha formulation containing from 2 to 75 mM methionine and the application US 2003/0104996 describes formulations of erythropoietin (EPO) or hyperglycosylated erythropoietin (NESP) the degradation of which is limited by the addition of a quantity of methionine ranging up to 50 mM.

Nevertheless, this alternative for preventing oxidation is not completely satisfactory.

Thus, the quantity of methionine to be combined with the protein or enzyme in order to guarantee oxidation stability for the latter, may of course vary depending on the nature of the protein, its concentration, the pH and other elements of the formulation. On the other hand, when this protein or this peptide is already utilized in a form combined with an amphiphilic polymer as defined previously, the efficacy of the methionine can be altered. Finally, in the particular case of an implant or a gel obtained from such an amphiphilic polymer, the added methionine may lose its efficacy as it cannot be distributed homogeneously and permanently in the implant or the gel.

The present invention aims precisely to make up for the abovementioned insufficiencies.

More precisely, an objective of the present invention is to propose a novel type of amphiphilic polymer, capable of being utilized as a vehicle of an active ingredient, and more particularly of protein, peptide or enzyme type which is naturally sensitive to an oxidation phenomenon, and which is precisely able to guarantee to such an active ingredient a stability against this phenomenon.

Another essential objective of the present invention is that these polymers moreover have a tendency to combine and can therefore be used as vectors of active ingredients, and therefore exhibit an ability to combine easily with numerous active ingredients and release them in vivo.

As a result, according to one of its aspects, the present invention relates to an amphiphilic polymer comprising a hydrophilic backbone, characterized in that said backbone bears at least one methionine side group or one of its derivatives and hydrophobic side groups distinct from said methionine or one of its derivatives.

Preferably, the methionine group or groups and/or hydrophobic groups are arranged randomly.

According to another of its aspects, the present invention relates to the use of a polymer as defined above for the vectorization of active ingredients, AIs, in particular proteins or peptides which are sensitive to oxidation.

According to yet another of its aspects, it relates to nanoparticles comprising at least one polymer according to the invention, in combination or not in combination with an active ingredient.

According to yet another of its aspects, the present invention relates a composition containing a polymer according to the invention and an active ingredient, organized or not organized in the state of nanoparticles.

In the context of the present description, the term “methionine” is, unless otherwise specified, used to denote either the methionine residue or a derivative of the latter in particular as defined hereafter.

Within the meaning of the invention and in the whole of the present disclosure, the terms “combination” or “combine” used to qualify the interaction between one or more active ingredients and the polymers considered according to the invention mean, in particular, that the active ingredient or ingredients are linked to the polymer(s) in particular by a hydrophobic and/or electrostatic interaction, and/or are solubilized by the polymer or polymers.

Within the meaning of the present invention, the term “side” means that the hydrophobic and methionine groups are arranged so as to appear as pendant groups or also “grafts” on the linear backbone.

Within the meaning of the present invention, by “random” is meant that the monomer unit or units of the amphiphilic polymer of the invention bearing a methionine group or groups and that (those) bearing a hydrophobic group or groups are distributed in an irregular manner within the hydrophilic backbone, independently of the nature of the adjacent units.

As is clear from the following, it is to the credit of the applicant company that it has developed a novel family of amphiphilic polymers, able to form stable nanoparticulate systems and the hydrophilic backbone of which comprises on the one hand one or more methionine side grafts, and on the other hand several hydrophobic side groups distinct from methionine.

The document WO 2008/094144 already describes a hydrophilic polymer of polyaspartic type bearing methionine and/or cysteine grafts. This type of polymer is however devoid of hydrophobic grafts distinct from methionine as required according to the invention. Moreover, it is proposed there only for the purposes of surface treatment of nanoparticles. This document is therefore not at all concerned with the antioxidant activity of the methionine and still less with the potential valorization of the latter for purposes of developing a polymeric vehicle capable of preserving an oxidation stability of active ingredients precisely sensitive to this phenomenon.

Against all expectation, the inventors have noted that the presence of methionine in the state of graft(s) on an amphiphilic polymer makes it possible to give said polymer a significant antioxidant activity, without moreover affecting its ability, on the one hand to combine with an active ingredient and, on the other hand, to be organized in the state of nanoparticles when brought into contact with an aqueous medium.

Moreover, the polymer considered according to the invention, advantageously lends itself to adjustment in terms of antioxidant activity with respect to its methionine grafting rate. This possibility of modifying the methionine grafting rate is particularly significant in view of the fact that it makes it possible to adjust the antioxidant activity of the polymer according to the invention as a function of the oxidation sensitivity of the protein or of the peptide having to be stabilized by said polymer.

Amphiphilic Polymer

Hydrophilic Backbone

As specified above, the amphiphilic polymer considered according to the invention has a hydrophilic backbone.

Advantageously, the polymers considered according to the invention have a polymeric backbone which is soluble in water, in particular at a pH comprised between 5 and 8.

This backbone can in particular be chosen from a polymer or copolymer belonging to the family of the polyamino acids, polysaccharides, polyacrylates or polymethacrylates.

The following are therefore quite particularly suitable,
polyamino acids such as poly(glutamic acid) (of alpha or gamma type), poly(aspartic acid) (of alpha or alpha/beta type), the polylysines (of alpha type) or copolymers formed by combinations of these same amino acids,
poly(acrylic acid) or poly(methacrylic acid), and
the polysaccharides such as dextran or one of its derivatives such as carboxymethyl dextran or hydroxyethyl dextran or pullulans.

According to a particular embodiment, the hydrophilic backbone of the amphiphilic polymer of the invention is a polyamino acid chosen from poly(glutamic acid), poly(aspartic acid), the polylysines, or their copolymers.

A certain number of polymers which can be used according to the invention, for example poly(alpha-L-glutamic acid), poly(alpha-D-glutamic acid), poly(gamma-L-glutamic acid) and poly(alpha-L-lysine) type of variable masses are commercially available.

The polymers capable of forming the hydrophilic backbone of the amphiphilic polymers of the invention can moreover be obtained by methods known to a person skilled in the art.

For example, the synthesis of a sodium polyglutamate of alpha type can be carried out via the polymerization of N-carboxy-amino acid anhydrides (NCAs), as described, for example, in the article “Biopolymers, 1976, 15, 1869 and in the book by H. R. Kricheldorf “alpha-Aminoacid-N-carboxy Anhydride and related Heterocycles” Springer Verlag (1987). The derivative of NCA is preferably NCA-Glu-O—R3 (R3=methyl, ethyl or benzyl). The polymers are then hydrolyzed under appropriate conditions in order to obtain the polymer in its acid form. These methods are based on the description given in the applicant company's patent FR 2 801 226.

These polymers have as activatable groups carboxylic, amine or alcohol functions. Their grafting can therefore be envisaged without difficulty.

Generally, the linear polymers with a hydrophilic backbone are grafted at the same time or sequentially by the hydrophobic group or groups and methionine(s) or methionine derivative(s).

Hydrophobic Groups and Methionine Groups

According to an essential characteristic of the invention, the polymers of the invention are grafted with at least one methionine group (MG) and with pendant hydrophobic groups which are different from methionine (HG).

Methionine Group (MG)

Within the meaning of the present invention, by the expression “methionine derivative” is meant more particularly the substitution derivatives, in particular at the level of the amine or carboxylic function of the methionine.

These are for example methionine amide or methionine ethyl ester.

These compounds are more particularly advantageous for carrying out a coupling reaction via the amine function of the methionine.

It is obvious that the methionine comprises two reactive functions—carboxylic acid and primary amine—and depending on the coupling reaction implemented, one or other of the functions must be protected.

The methionine which can be used in the polymers of the invention can be of L, D configuration or a racemic mixture.

Depending on the type of grafting, the linking function is of amide or ester type. The methionine group, once grafted onto the polymer, generally has one of the three structures below:

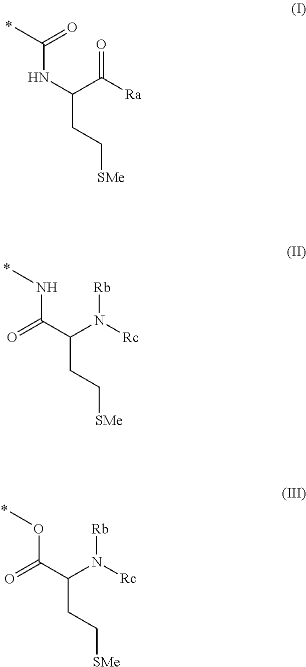

in which:
Ra represents a hydroxy group (optionally deprotonated), NH₂, OMe, OEt, NHCH₃ or N(CH₃)₂,
Rb and Rc represent independently a hydrogen atom, a methyl or an ethyl,

with, when one of the Rb and Rc groups is a hydrogen atom, then the other group represents a C₁ to C₆ acyl group, and the configuration of the methionine can be L, D or a racemic mixture.

Hydrophobic Groups (HG)

The hydrophobic groups are, in practice and without this being limitative, chosen from the group comprising the alcohols and the amities, these compounds being able to be easily functionalized by a person skilled in the art, and their grafting implementing reactions analogous to those required for the methionine derivative.

According to a preferred characteristic, the hydrophobic group (HG) comprises from 5 to 30 carbon atoms.

These hydrophobic groups (HG) are advantageously and judiciously selected from the group comprising:
the linear or branched C₅ to C₃₀ alkyls optionally comprising at least one unsaturation and/or at least one heteroatom,
the C₈ to C₃₀ alkylaryls or arylalkyls optionally comprising at least one unsaturation and/or at least one heteroatom,
and the C₁₀ to C₃₀ (poly)cyclics optionally comprising at least one unsaturation and/or at least one heteroatom.

More particularly, the hydrophobic groups (HG) can be, for example, groups chosen from the group comprising:
dodecanoxy, tetradecanoxy, hexadecanoxy, octadecanoxy, oleyloxy, tocopheryl or cholesteryl, aminohexyl, aminohexadecyl and aminooctadecyl,
lauryl, myristyl, palmityl and stearyl,
a hydrophobic amino acid such as leucine, valine, phenylalanine, tryptophan or tyrosine or one of their derivatives.

When the hydrophobic group is an amino acid, it can be a derivative corresponding to one of the structures below (IV), (V) or (VI) in which Ra, Rb and Rc correspond to the definitions given previously and Rd corresponds to an amino acid residue depending on the type of polymer and the grafting reaction implemented.

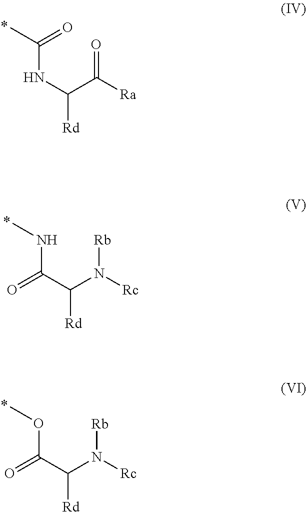

Alternatively, the methionine(s) or methionine derivative(s) and/or the hydrophobic groups can be linked to the polymeric backbone via a spacer making it possible to link them to the polymer chain. This spacer is advantageously divalent and belongs to the group comprising in particular the amino acid units, amino alcohol derivatives, diamine derivatives, diol derivatives and hydroxy acid derivatives.

According to a particular embodiment of the invention, the hydrophilic backbone of the amphiphilic polymer is a sodium poly(L)glutamate, and the hydrophobic group a tocopheryl group of synthetic origin and, preferably, the methionine derivative is methionine amide or methionine ethyl ester.

As a result, the preferred amphiphilic polymers of the invention can be diagrammatically represented by the fact that they are formed by a sequence of the following general structure (I):

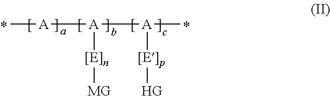

in which:
A represents monomeric units of the hydrophilic polymer chain,
MG represents methionine or one of its derivatives,
HG represents a hydrophobic group and
E and E′ represent a spacer group with n and p representing independently of each other 0 or 1,

with a, b and c being integers different from zero,

the hydrophobic groups HG and the methionine groups mg being distributed randomly.

Advantageously, the hydrophobic groups molar percentage is represented by the ratio c/(a+b+c) and the methionine groups molar percentage is represented by the ratio b/(a+b+c).

The molar grafting rate of hydrophobic units in the polymers according to the invention advantageously varies from 2 to 30%, and preferably from 5 to 20%.

The molar grafting rate of methionine unit(s) in the polymers according to the invention, varies from 0.5 to 20%.

As regards the a/a+b+c molar ratio, it varies between 40 and 97.5%.

Advantageously, the polymers according to the invention have a molar mass by weight which is situated between 2,000 and 200,000 g/mole, and preferably between 5,000 and 100,000 g/mole.

According to another variant, the polymers according to the invention can also bear at least one graft of polyethylene glycol type.

Preferably, the molar mass of the polyethylene glycol is from 1,000 to 5,000 Da. The polyethylene glycol type group can be represented diagrammatically according to one of the following structures:

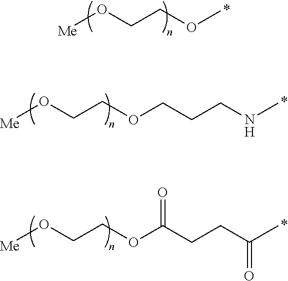

Preferably, the polyethylene glycol molar grafting percentage varies from 1 to 10%. This unit may or may not be directly linked to the hydrophilic backbone of the polymer according to the invention.

Method for the Preparation of the Amphiphilic Polymer

As regards the methionine, the grafting of its amine function can easily be carried out by coupling of the latter with a carboxylic function present on the amphiphilic polymer backbone, in the presence of a coupling agent such as a carbodiimide and a catalyst such as dimethylaminopyridine. This reaction can be carried out in an organic solvent or in aqueous phase. In the second case, a water-soluble carbodiimide is preferably used. The carboxylic function can be left free or protected by a group forming an ester or amide bond.

Thus, when the polymer possesses amine or alcohol functions, the grafting of the methionine is then done via the carboxylic function, having protected the amine function of the methionine with an acyl group or by dimethylation beforehand. These reactions are well-known to a person skilled in the art and the book by g. Hermanson (Bioconjugate Techniques 2^{nd} edition 2008, Elsevier), for example, describes these methodologies.

For their part, the pendant hydrophobic groups (HG) can be linked to the polymer via an amide, ester, carbonate or carbamate function, depending on the nature of the activatable function of the polymer and that of the graft.

Preferably, the bond is of amide or ester type as for the methionine derivative. In this case, the grafting of the methionine derivative and of the hydrophobic group can be done simultaneously.

In order to obtain a polyamino acid grafted with hydrophobic groups, a coupling reaction is carried out between the hydrophobic group comprising a reactive amine or alcohol function and the polymer comprising carboxylic acid functions in the presence of a condensation agent such as diisopropylcarbodiimide and a catalyst such as dimethylaminopyridine. This reaction can take place in a solvent such as dimethylformamide (DMF), dimethyl sulphoxide (DMSO) or N-methylpyrrolidone (NMP). Ideally the grafting of the methionine is carried out at the same time. The bonds formed are ester or amide bonds.

The coupling reagents such as the chloroformates can also be used for the formation of amide bonds (see for example the work by Bodanszky “Principles of Peptide Synthesis” Springer Verlag 1984 for examples of coupling agents). The grafting rate is chemically controlled by the stoichiometry of the constituents and reagents and/or the reaction time.

In the case of a polymer comprising amine groups such as polylysine or dextran also comprising alcohol groups, the hydrophobic groups considered then bear carboxylic acid groups. The bonds formed after coupling are in particular ester, amide, carbonate or carbamate bonds.

By way of examples, we describe hereafter several types of amphiphilic polymers which can comprise both hydrophobic groups and methionine groups mg, and the manner of obtaining them according to procedures described in the literature.

As regards a pullulan type polymer grafted with cholesterol, this can be synthesized according to the procedure described in the U.S. Pat. No. 6,566,516. A derivative having a reactive isocyanate is then prepared by reacting a diisocyanate with a methionine the acid function of which is protected by an —NH₂ (amide) or an —OMe (ester). This procedure is standard and is described in Example 1 of the same patent. The grafting can be carried out according to the method described in Example 2.

Finally, a polymer of dextran type grafted with a lauroyl group and a methionine derivative can be obtained by reacting the dextran polymer with the acid chloride of lauric acid and the acid chloride of N-acetylmethionine in N-methylpyrrolidone. The procedure for obtaining dextran grafted with the lauroyl group is described in the U.S. Pat. No. 5,750,678 (Example 1).

Similarly, a polyacrylate comprising stearylamine and methionine amide can be prepared according to the procedure described in the U.S. Pat. No. 6,607,714 by having the methionine amide present in the desired quantity during the grafting stage.

A poly(gamma)glutamate comprising a phenylalanine ester and a methionine ester can also be prepared according to the protocol described by Matsusaki et al. Chem. Letters 2004, 33, 398-399. A mixture of a methionine ethyl ester and a phenylalanine ethyl ester in the presence of a water-soluble carbodiimide can be simultaneously grafted onto a poly(gamma-glutamic acid) in water.

Finally, a polylysine comprising a stearoyl group, N-acetylmethionine and a polyethylene glycol chain can be prepared according to the procedure described by Brown et al., Bioconjugate Chem 2000, 11, 880-891. In this embodiment, the methionine derivative to be grafted onto the polylysine contains the hydroxysuccinimide group on the carboxylate and an acetyl on the amine function.

Active Ingredient (AI)

According to a preferred embodiment of the invention, the active ingredients, AIs, capable of being combined with a polymer according to the invention are chosen from the proteins, or peptides, i.e. from AIs sensitive to the phenomenon of oxidation.

More particularly, these are peptides or proteins comprising at least one methionine in their sequence. In fact, the methionines are particularly subject to oxidation.

In this category, the proteins such as growth hormone, the interferons, the coagulation factor proteins such as factor VII, factor VIII and factor IX, the EPOs, the GCSFs and monoclonal antibodies are known to be easily oxidized. These proteins can optionally comprise at least one polyethylene glycol chain.

The techniques for combining one or more AIs with an amphiphilic polymer according to the invention are described in particular in the U.S. Pat. No. 6,630,171.

They consist of incorporating at least one active ingredient in the liquid medium containing polymers of the invention loaded with, or combined with, one or more active ingredient(s). This incorporation can be carried out as follows: putting the polymer in aqueous solution, then addition of the active ingredient, in solid form or in aqueous solution.

Preferably, the active ingredient is chosen from the group comprising: the proteins, glycoproteins, proteins linked to one or more polyalkylene glycol chains [preferably polyethylene glycol (PEG): “PEGylated proteins”].

Nanoparticles

Advantageously, the polymers considered according to the invention in combination or not in combination with an AI, in particular as defined above, are capable of spontaneously forming nanoparticles when they are dispersed in an aqueous medium with a pH ranging from 5 to 8, in particular in water.

Generally, the formation of nanoparticles is due to a self-association of a multitude of polymer chains with segregation of the hydrophobic groups in nanodomains. A nanoparticle can contain one or more hydrophobic nanodomains.

The size of the polymer nanoparticles can vary from 1 to 1,000 nm, in particular from 5 to 500 nm, in particular from 10 to 300 nm, and more particularly from 10 to 200 nm, or even from 10 to 100 nm.

Applications

As specified above, the amphiphilic polymers of the invention may be used in several ways depending on the nature of the hydrophobic groups, their molar percentage of methionine and their degree of polymerization. The methods of shaping a polymer for the encapsulation of an active ingredient in the various forms referred to by the invention are known to a person skilled in the art.

For more details, these few particularly relevant reference works can, for example, be consulted:
formulation of a protein with a polyamino acid comprising hydrophobic groups in the form of nanoparticles or microparticles: WO 00/30618, WO 2005/051418, WO 2007/141344, WO 2008/025425, WO 2008/135561,
formulation of a protein with a pullulan comprising a hydrophobic group such as cholesterol: U.S. Pat. No. 6,566,516.

According to another of its aspects, the invention relates to a composition, in particular pharmaceutical, cosmetic, dietetic or phytosanitary, comprising at least one polymer as defined above and at least one active ingredient, in particular subject to oxidation. This is more particularly a protein, a peptide or an enzyme sensitive to oxidation.

In particular, the protein, peptide or enzyme contains at least one methionine in its sequence.

According to an embodiment variant, this polymer, combined or not combined with an AI, can be in the state of nanoparticles.

According to an embodiment, the composition of the invention can be presented in the form of a gel, a solution, a suspension, an emulsion, micelles, nanoparticles, microparticles, an implant, a powder, a suspension, a lyophilisate or a film, and preferably in the form of nanoparticles, microparticles, gels or films.

Advantageously, it is able to ensure a release profile of said active ingredient regulated as a function of time.

According to one of its particularly preferred forms, the composition, loaded or not loaded with active ingredient(s), is a stable colloidal suspension of nanoparticles and/or microparticles and/or micelles in an aqueous phase.

Microparticles can be obtained by various methods such as coacervation in the presence of an aggregation agent (divalent or trivalent ions or polyelectrolytes), precipitation by change of pH or of the ionic force, extraction/evaporation, by atomization or by freeze-drying.

The composition according to the invention, when it is pharmaceutical, can be administered by oral, pulmonary, parenteral, nasal, vaginal, ocular, sub-cutaneous, intravenous, intramuscular, intradermal, intraperitoneal, intracerebral or buccal route.

According to another embodiment, the composition can optionally contain an excipient for the adjustment of the pH and/or of the osmolarity and/or for improving the stability and/or as an antimicrobial agent. These excipients are well-known to a person skilled in the art (refer to the work: Injectable Drug Development, P. K. Gupta et al. Interpharm Press, Denver, Colo. 1999).

The examples and figure which follow are presented by way of illustration and are non-limitative of the field of the invention.

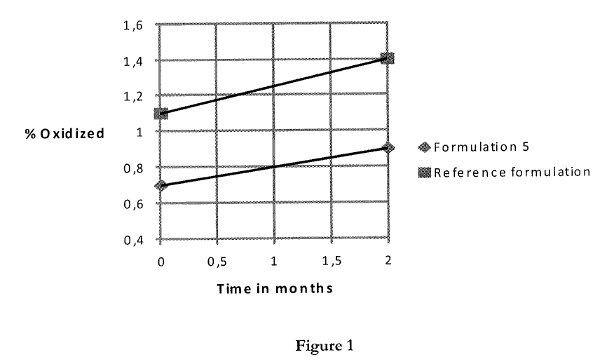

FIG. 1 Graphic representation of oxidized protein rates measured at T0 and after 2 months T(2 months) at 5° C., according to example 5, for a formulation of interferon alpha-2b formulated with the polymer of example 1 according to the invention (formulation 5), and for a comparative formulation of interferon alpha-2b formulated with methionine (reference formulation).

### EXAMPLE 1

### Synthesis of a Sodium Polyglutamate Comprising Grafts of Alpha-Tocopherol and Methionine

A polyglutamate statistically grafted with 5% racemic alpha-tocopherol is synthesized according to the method described in the Application WO 03/104303 (Example 1). 5 g of the polymer in its polyacid form is dissolved in 77 mL of DMF at 70° C. After dissolution of the solid, the solution obtained is cooled down to 0° C. 108 mL of isobutyl chloroformate and 92 mL of N-methylmorpholine are added, then the white suspension obtained is stirred at 0° C. for 10 minutes. In parallel, 536 mg of methionine ethyl ester hydrochloride (MetOEt.HCl) are dissolved in 8.2 mL of NMP then 349 mL of triethylamine are added. This mixture is added to the suspension of activated polymer, and the reaction mixture is stirred at 0° C. for 1 hour. After addition of 1 mL of concentrated HCl (35%) then 50 mL of water, the mixture is neutralized with 1N soda. The solution obtained is diafiltered against salt water (0.9%), then water, and concentrated to a volume of approximately 150 mL. The molar grafting rate of the methionine ethyl ester, determined by HPLC after acid hydrolysis of the polymer, is 1.7% of the monomer units.

### EXAMPLE 2

### Synthesis of a Sodium Polyacrylate Comprising Grafts of Alpha-Tocopherol and Methionine

### Step 1: Purification of Commercial Polyacrylic Acid (Degacryl 4779L)

75 g of a DEGACRYL 4779L solution (by Evonik) are dissolved with 1425 g of water, then diafiltered against 8 volumes of water. The solution obtained is further freeze-dried. The average molecular mass Mn, measured by steric exclusion chromatography, is 33.6 kDa in PMMA (polymethylmethacrylate) equivalent and the polydispersity index is 2.4.

### Step 2: Synthesis of the Alanine α-tocopherol (AlaVE) Ester

22 mL of N,N′-Diisopropylcarbodiimide (DIPC) are added drop by drop to a solution of 21.1 g N-Boc alanine, 40 g α-tocopherol and 0.57 g dimethylaminopyridine (DMAP) in 400 mL of dichloromethane. After stirring at 20° C. for 22 h, the reaction mixture is successively washed with a 0.1 N HCl solution, water, a 5% sodium bicarbonate solution and finally water. The organic phase is evaporated to dryness and the oil obtained is dissolved in 400 mL of 4 M HCl solution in dioxane. After 4 h stirring at room temperature, the reaction mixture is evaporated to dryness and crystallized in ethanol. The AlaVE hydrochloride (33.8 g of white powder) thus prepared is analyzed by proton NMR in CDCl₃ and shows a spectrum in accordance with its chemical structure.

### Step 3: Grafting of AlaVE and Methioninamide on Purified Polyacrylic Acid

2.25 g of AlaVE are dissolved in 58 mL of DMF and 0.58 mL of triethylamine. In parallel, 19 mg of methioninamide hydrochloride are dissolved in 2 mL of DMF and 0.27 mL of triethylamine. 5 g of purified DEGACRYL (step 1) are dissolved in 125 mL of N,N-dimethylformamide (DMF) and 0.25 g of 4-dimethylaminopyridine (DMAP). This solution is cooled at 15° C. and the AlaVE/triethylamine suspension, the MetNH₂/NEt₃ solution, and 1.66 mL of N,N′-Diisopropylcarbodiimide (DIPC) are successively added. The reaction mixture is stirred one night at 15° C. After addition of an HCl 35% solution (0.56 mL) diluted in 6 mL of DMF, the reaction mixture is neutralized with 1 N soda in 200 mL of water. The solution obtained is purified by diafiltration and concentrated until a volume of approximately 200 mL.

The percentage of AlaVE determined by proton NMR in TFA-d is 6% and the percentage of grafted methioninamide determined by HPLC after hydrolysis is 5.5% of the monomer units.

### EXAMPLE 3

### Synthesis of a Sodium Polyglutamate Comprising Grafts of Octadecylamine and Methionine

5 g of a polyglutamate having a DP 100 are dissolved at 80° C. in 110 mL of DMF. After dissolution of the solid, a solution of 95 mg of 4-dimethylaminopyridine (DMAP) in 1 mL of DMF is added, the mixture is stirred at 80° C. for 18 h, then cooled at 15° C. 220 μL of triethylamine are added to a solution of 286 mg of methioninamide hydrochloride in 5 mL of DMF and the solution is stirred at room temperature. A solution of 1.04 g of octadecylamine in 11 mL of DMF, the preceding solution, 189 mg of DMAP in 1 mL of DMF, and 1.26 mL of diisopropylcarbodiimide (DIPC) are successively added to the solution of polyglutamate in DMF. The reaction mixture is stirred at 15° C. for 24 h, then the reaction is stopped by adding a solution of 0.3 mL of concentrated HCl (35%), 0.3 mL of water and 5 mL of DMF. The solution is poured into 500 mL of water and neutralized with 1 N soda. The solution obtained is diafiltered against salt water (0.9%) then against water, and concentrated. The molar grafting rates of octadecylamine and methioninamide, determined by proton NMR in TFA-d, are respectively 10 and 4% of the monomer units.

### EXAMPLE 4

### Study of the Stability of Growth Hormone Formulated with the Polymer of Example 1

The formulation is prepared by simple mixture of a polymer solution 1 adjusted in pH (HCl or NaOH) and in osmolality (NaCl) to approximately pH 7.0 and 300 mOsm/Kg and of a protein solution, in order to obtain a final growth hormone concentration of 0.7 mg/mL and a polymer 1 concentration of 22 mg/mL. Under these conditions, the formulation containing the polymer 1 has an equivalent methionine concentration of approximately 2.4 mM. The polymer solution was filtered through a 0.2 μm filter beforehand. The formulation finally obtained is stirred overnight at ambient temperature then divided, one part being placed in a refrigerator at 5° C. The oxidized growth hormone ratio in the samples is measured by liquid chromatography (HPLC) according to the following conditions: Column Symmetry 300 C18 (Waters; 150×4.6 mm; 3.5 μm), flow rate: 0.8 mL/min, UV detection: 220 nm, temperature of the column 55° C., potassium phosphate buffer 25 mM pH=6.5/Propanol−1 as eluent.

Two methionine residues are oxidation-sensitive in the growth hormone: the methionine in position 14 and the methionine in position 125. These two degradation products are taken into account in the quantification of the oxidation.

The levels of oxidized protein measured at T0 and after 2 months T(2 months) at 5° C. are given in the following Table 1:

TABLE 1

| | T0 | T (2 months) at 5° C. |
|---|---|---|
| Polymer 1 | 2.7 | 3.3 |
| Protein without polymer | 3.3 | 10.7 |

### EXAMPLE 5

### Study of the Stability of the Interferon Alpha 2b Formulated with the Polymer of Example 1

The formulation (formulation 5) is prepared by simple mixture of a polymer solution of Example 1 adjusted in pH and in osmolality (to approximately pH 6.5 and 300 mOsm/Kg) and of a protein solution, in order to obtain a final interferon alpha 2b concentration of 0.3 mg/mL and a polymer concentration of 22 mg/mL. Under these conditions, the formulation containing the polymer 1 has an equivalent methionine concentration of approximately 2.4 mM. The polymer solution was filtered through a 0.2 μm filter beforehand. The formulation finally obtained is stirred overnight at ambient temperature then placed in a refrigerator at 5° C.

For the sake of comparison, a solution of the protein having an equivalent methionine concentration is prepared in similar conditions (reference formulation). The oxidized form ratio (corresponding to the oxidation of the methionine in position 111) is measured by liquid chromatography (HPLC), according to the following chromatographic conditions: Column YMC C30 (Interchim; 250×4.6 mm; 3 μm), flow rate: 1 mL/min, fluorimetric detection: excitation at 280 nm and emission at 340 nm, temperature of the column 20° C., water/acetonitrile/TFA as eluent.

The ratios of oxidized protein measured at T0 and after 2 months T(2 months) at 5° C. are shown on FIG. 1.

From Examples 4 and 5 it can be concluded that the use of a polymer according to the invention makes it possible to reduce more efficiently the rate of protein oxidized during the formulation and/or to maintain a low rate over time.

### EXAMPLE 6

### Preparation of Microparticles from the Polymer of Example 1 and a Polymer of the Prior Art not Containing any Methionine Binded

For some applications, the fabrication of a formulation containing microparticles of polymers and an active principle is preferred (for example in the application WO 2007/141344 by the applicant). A microparticulate formulation is prepared according to example 18 of the application WO 2007/141344, in other words, starting from a polymer PO polyglutamate grafted with alpha-tocopherol, IFN and methionine in a free state.

A microparticulate formulation is prepared in similar conditions starting from IFN and the polymer of example 1 according to the invention, comprising alpha-tocopherol grafts and methionine grafts.

The amount of methionine in the microparticles is measured. The results are given in Table 2 below.

TABLE 2

| | Polymer | Initial methionine | Final methionine |
|---|---|---|---|
| | Concentration | concentration | concentration after |
| | (mg/mL) | (mM) | formulation (mM) |
| Polymer of | 9 mg/mL | 1 mM | 1 mM (no loss) |
| example 1 | | | |
| Polymer of the | 15 mg/mL | 2.4 mM | 0.1 mM (96% loss) |
| prior art | | | |

This demonstrates that there is an important loss in methionine with the microparticles formulation according to the application WO 2007/141344, which is not observed when the polymer 1 according to the invention is used. Moreover, the use of a polymer according to the invention enables a homogeneous distribution of the methionine in the particles, whatever their size.

1. Amphiphilic polymer comprising a hydrophilic backbone, wherein said backbone comprises at least one side group of methionine or methionine derivative, and comprises hydrophobic side groups distinct from said methionine or one of its derivatives.

2. Polymer according to claim 1, in which the at least one side group of methionine or methionine derivative and hydrophobic side groups are arranged randomly.

3. Polymer according to claim 1, wherein the hydrophilic backbone is a polymer or copolymer selected from the group of: polyamino acids, polysaccharides, polyacrylates and polymethacrylates.

4. Polymer according to claim 1, wherein the hydrophilic backbone is a polyamino acid selected from the group of: poly(glutamic acid), poly(aspartic acid), the polylysines and their copolymers.

5. Polymer according to claim 1, wherein said polymer has a methionine molar grafting rate varying from 0.5 to 20%.

7. Polymer according to claim 1, wherein the at least one side group of methionine or methionine derivative possess(es) one of the following structures: 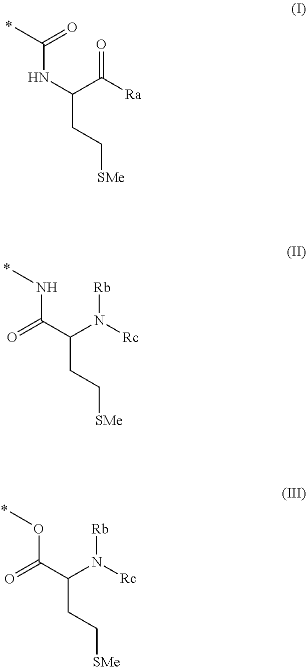
in which:
Ra is selected from the group consisting of: a hydroxy, NH₂, OMe, OEt, NHCH₃ and N(CH₃)₂ group,
Rb and Rc represent independently a hydrogen atom, a methyl or ethyl;
with when one of the groups Rb and Rc is a hydrogen atom, then the other group represents a C₁ to C₆ acyl group, and the configuration of the methionine can be L, D or a racemic mixture.

7. Polymer according to claim 1, said polymer comprising a hydrophobic groups molar grafting rate varying from 2 to 30%.

8. Polymer according to claim 1, wherein the hydrophobic group is selected from the groups comprising:
the linear or branched C₅ to C₃₀ alkyls,
the linear or branched C₅ to C₃₀ alkyls comprising at least one unsaturation and/or at least one heteroatom,
the C₈ to C₃₀ alkylaryls or arylalkyls,
the C₈ to C₃₀ alkylaryls or arylalkyls comprising at least one unsaturation and/or at least one heteroatom,
the C₁₀ to C₃₀ (poly)cyclics, and
the C₁₀ to C₃₀ (poly)cyclics optionally comprising at least one unsaturation and/or at least one heteroatom.

9. Polymer according to claim 1, wherein said hydrophilic backbone is a sodium poly(L)glutamate and the hydrophobic group a tocopheryl group of synthetic origin.

10. Polymer according to claim 1, wherein said methionine derivative is methionine amide or methionine ethyl ester.

11. Polymer according to claim 1, wherein said polymer has a molar mass by weight ranging from 2,000 to 200,000 g/mole.

12. Polymer according to claim 1, wherein said polymer comprises at least one graft of polyethylene glycol type.

13. Polymer according to claim 1, wherein said polymer is capable of spontaneously forming nanoparticles when dispersed in an aqueous medium with a pH ranging from 5 to 8.

14. Nanoparticles of a polymer according to claim 1, wherein the size of said polymer is between 1 and 1000 nm.

15. Composition comprising a polymer according to claim 1 and at least one active ingredient selected from the group consisting of: oxidation-sensitive protein and oxidation-sensitive peptide.

16. Composition according to claim 1, wherein said protein or peptide contains at least one methionine in its sequence.

17. Composition according to claim 15 wherein said composition is in the form of nanoparticles, microparticles, gels or films.

18. Composition according to claim 15, wherein the release profile of said active ingredient is regulated as a function of time.
